(19) United States
(12) Patent Application Publication
(10) Pub. No.: US 2003/0007996 A1
(43) Pub. Date: Jan. 9, 2003

(54) Therapies for the prevention and treatment of diabetes and obesity

(76) Inventors: Terry E. Graham, Guelph (CA); Erik A. Richter, Gentofte (DK); Farah S.L. Thong, Guelph (CA); Lindsay E. Robinson, Guelph (CA)

Correspondence Address: BARBARA KITCHELL, Akerman, Senterfitt Eidson, P.A., Suite 400, 222 Lakeview Avenue, West Palm Beach, FL, 33401, US

(21) Appl. No.: 10/158,373

(22) Filed: May 30, 2002

Related U.S. Application Data
(60) Provisional application No. 60/294,464, filed on May 30, 2001.

(51) Int. Cl. A61K 47/00

(52) U.S. Cl. 424/439

(57) ABSTRACT

A method for the prevention and treatment of diabetes and obesity by a system of health management promoting a caffeine reduced diet and the use of adenosine analogues and adenosine receptor agonists. Methods for diet plans and labelling are disclosed. Use of decaffeinated coffee is promoted.

### CROSS REFERENCE TO RELATED APPLICATION

The present application claims the priority of U.S. provisional patent application No. 60/294/464, filed May 30, 2001, and entitled “Therapies for the Prevention and Treatment of Diabetes.”

### FIELD OF THE INVENTION

The field of the present invention is human physiology. The present invention relates to the prevention and treatment of diabetes and obesity by a system of health management promoting a reduced caffeine diet and the use of adenosine, adenosine analogues, derivatives, conjugates thereof and adenosine receptor agonists.

### BACKGROUND OF THE INVENTION

Diabetes is a condition characterized by the body's inability to transport glucose from the blood into adipose or skeletal muscle cells. This results in glucose build up in the blood. Insulin is the key hormone that regulates glucose uptake in the body. Type 2 diabetics either do not make enough insulin or their cells are insensitive to it. Type 1 diabetics do not make insulin and have to administer it to their bodies.

It is estimated that at least 120 million people worldwide are suffering from Type 2 diabetes and this is predicted to almost double in our current decade (Shaw et al., 2000). This is attributed to aging populations, increases in obesity together with sedentary lifestyles and poor nutritional habits. As such it is clear that adopting a positive lifestyle would both reduce the probability of developing Type 2 diabetes and/or delay the onset and modify the severity. In Canada by 2000 there are expected to be 2.2 million diabetic patients and this should increase to 3 million by 2010 (Meitzer et al, 1998; Tan and MacLean 1995). About 90% of these patients are expected to be Type 2 diabetics. Diabetes is a major concern not only because of its well known links with cardiovascular disease but also because of its increased risks of blindness, kidney disorders, peripheral neuropathies and vascular disorders. It is believed that only about 50% of Type 2 diabetics are diagnosed; this together with the wide ranging complications make it difficult to establish the total health costs and impact on the quality and quantity of life. The underlying etiology of Type 2 diabetes is not resolved, but it is clear that a negative lifestyle in nutrition and exercise are key factors.

Type 1 diabetics must administer insulin to themselves their entire life time. Currently, experimental studies are underway where beta islets cells are transplanted into Type 1 diabetics. Type 2 diabetics must control their diet, are encouraged to lose weight and to exercise. Often they ingest drugs that increase their cells' sensitivity to insulin (Metformin™ and Thiazolidinedione™). In addition, certain drugs stimulate the pancreas to release insulin (sulfonylureas and benzoic acid derivatives).

Skeletal muscle is a key target tissue for glucose metabolism and insulin action. Skeletal muscle is perhaps the most critical tissue in glucose management, it contains approximately 80% of the body's carbohydrate stores and is the largest tissue under insulin regulation. Any changes in the regulation of muscle glucose metabolism can have a profound influence on the metabolism and health of the body (Graham et al., 1999; Jequier and Tappy, 1999). For example, muscle glycogen accounts for 35% of the glucose storage following a mixed meal (Taylor et al., 1993) and 50-90% of the glucose disposal during an oral glucose tolerance test (OGTT) (Brundin et al 1996; Cortright and Dohm, 1997). In contrast, chronic exposure to high levels of exogenous carbohydrate result in a decline in insulin sensitivity in muscle (Laybutt et al., 1997). Muscle normally ‘buffers’ and stores ingested glucose, but habitual, excessive carbohydrate intake can impair this function, and for Type 2 diabetes, can impair glycemic control.

Glucose is taken into adipocytes and muscle cells via transporters. The main transporter is GLUT 4 and normally most of the cell's GLUT 4 is stored within the cell. Insulin and other metabolic signals can cause the GLUT 4 to translocate and become incorporated into the membrane. This results in greater glucose uptake by the cell. Obesity and/or a sedentary lifestyle are associated with a decreased ability of insulin to promote the incorporation of GLUT 4 transporters into the muscle sarcolemma, but the mechanisms by which insulin receptors, signaling and/or GLUT 4 translocation are changed are far from established (Cortight and Dohm, 1997).

Adenosine, a purine nucleoside, is a local metabolite produced in probably every tissue. It can be formed as a result of either ATP (adenosine triphosphate) or S-adenosylhomocysteine hydrolysis. This combined with a very short half life of approximately one second has resulted in it being designated as a ‘retaliatory metabolite’. Adenosine can be produced both intra- and extracellularly and binds to cell surface insulin receptors (Shryock and Belardinelli, 1997; Ralevic and Burnstock, 1998). These consist of 4 subsets; A1, A2a, A2b, and A3. A1 and A2 receptors are coupled to adenylate cyclase via Gi and Gs-proteins, respectively. While these can alter cAMP, A1 stimulation also activates phospholipase C, independent of cAMP and leads to formation of lipid by-products that activate protein kinases such as PKC (Ralevec and Burnstock, 1998). The function of adenosine receptors in tissues such as the central nervous system, heart and cardiovascular system and adipocytes, have been studied extensively. However, skeletal muscle has rarely been examined and there is no consensus regarding the roles of adenosine in skeletal muscle functions.

Adenosine is difficult to study in humans because it has a short half life. There are many adenosine receptor agonists and antagonists but most are not suitable for human consumption. However, trimethylxanthine (caffeine) and the dimethylxanthine, theophylline (found in tea) are nonselective adenosine receptor antagonists, are acceptable oral agents and, in physiological doses, most if not all of their actions on various tissues are due to this property. Plasma concentrations achieved by oral ingestion of 4-6 mg/kg of either these methylxanthines or large quantities of coffee or tea are 25-40 uM. Over 80% of North Americans consume caffeine daily and 20% consume greater than 5 mg/kg per day. Caffeine and theophylline are safe and reliable candidates to study the effects of adenosine receptor antagonism on glucose metabolism.

In adipose tissue it is very clear that A1 receptor antagonism inhibits insulin's ability to either take up glucose or to promote lipogenesis (Xu et al., 1998; Mauriege et al., 1995). It has been repeatedly demonstrated that adipocyte A1 receptor is linked to insulin signaling (Ralevic and Bumstock, 1998; Green, 1987) and Tagasuga et al, (1999) showed that adenosine augments insulin-stimulated glucose uptake of adipocytes in vitro. For example, the A1 receptor complement/function of adipocytes is adaptable by up- or down-regulation of receptor number and/or post-receptor signaling. Habitual exposure to antagonists caused an up regulation of receptors in some tissues but not in others (Green, 1987; Fredholm, 1995; Zhang and Wells, 1990). Rodent adipocyte A1 receptors and the post receptor mechanisms were unaffected by chronic caffeine administration (Fredholm, 1995). However, Mauriege et al., (1995) presented data suggesting that adipocytes from obese and lean women differed in their adenosine sensitivity. When they removed endogenous adenosine (analogous to blocking the A1 receptor) with adenosine deaminase prior to exposing femoral adipocytes in vitro to insulin, the adipocytes of obese women responded (there was a decrease in lipolysis), but there was no insulin response in the adipocytes from the lean (but not exercise-trained) women. These data suggest that adenosine receptor number or function can be altered by obesity and by an exercise-induced weight loss. However, the putative roles of adenosine with skeletal muscle has not been examined in such studies despite it being far more critical than adipose tissue in insulin resistance and glucose management.

In contrast to adenosine receptor antagonism decreasing insulin's actions in adipocytes, Challiss and coworkers (Budohoski et al., 1984; Challiss et al., 1984; Challis et al., 1992) have repeatedly found that this results in increased insulin sensitivity in rodent muscle in vitro. Only Xu et al. (1998) have been able to confirm this finding; others have reported that adenosine receptor antagonism causes marked decreases in muscle glucose uptake (Webster et al., 1996; Vergauwen et al., 1994; Han et al., 1998). However, even among these investigations there is controversy as to whether adenosine is important in both fast and slow twitch muscle, whether it plays a role in resting muscle or only during exercise, and even whether human tissue responds in a similar fashion to rodent muscle. The results observed in skeletal muscle are attributed to antagonism of A1 receptors. There are reports that rodent muscle has A2a receptors, but attempts to demonstrate A1 receptor or its mRNA have been negative (Dixon et al., 1996). There is no published data for human muscle, but there are reports (Challis et al., 1992; Webster et al., 1996) referring to unpublished findings that failed to show A1 receptors in human muscle.

Obese individuals have a decreased insulin sensitivity similar to diabetics, and it is clear that obesity is the dominant risk factor for developing Type 2 diabetes. There is one report that illustrated that adenosine-based responses of skeletal muscle were both critical and altered with obesity. Crist et al (1998) treated both normal and obese Zucker rats with an A1 antagonist for one week and then performed euglycemic-hyperinsulinemic clamps (there were no clamps performed on animals that were only acutely exposed to the antagonist). The lean animals that were exposed to the antagonist decreased whole body glucose disposal by 16%. Glucose uptake by the heart and liver were unaltered, while adipose tissue decreased its uptake by almost 50% and muscle decreased its uptake by 12-16%. However, absolute glucose uptake by muscle was much greater than that of adipose tissue as it represents a much larger mass. Assuming the rats were 40% muscle and 20% fat, 49% of the reduced glucose disposal could be attributed to muscle and 9% to adipose tissue. Mauriege et al (1995), Carey (2000) and Carey et al (1994) demonstrated that obesity and leanness are characterized by differences in adipocyte response to adenosine and this work by Crist et al (1998) showed that skeletal muscle of obese and lean animals differs in either the complement of adenosine A1 and/or A2a receptors or their interaction with insulin signaling and GLUT 4 translocation. Although these studies indicate there is a role for adenosine in glucose uptake and obesity, no experiments have been conducted with obese humans.

In summary, there is evidence that adenosine is an important regulator of insulin's actions in animal adipose and skeletal muscle cells. Further there is evidence that adenosine is an important regulator of insulin action on adipose cells in humans. While human muscle is a critical tissue for glucose management, very little is known regarding adenosine's actions on this tissue. Preliminary experiments have shown that caffeine inhibits insulin's ability to promote glucose uptake in skeletal muscles (Greer et al., 1998), and results in the body having to secrete more insulin in order to maintain glucose homeostasis in lean males (Battram et al., 1999). However, the sample numbers were low in these studies and these studies did not address whether chronic exposure to caffeine resulted in adaptations. Further, and more importantly, it is unclear how caffeine affects glucose uptake in muscle tissue of diabetics and the obese.

Given the high and growing frequency of obesity and Type 2 diabetes in our society, as well as the common ingestion of coffee, it is vital that these areas be studied in detail. It is also necessary to address the interaction between caffeine consumption and insulin administration in Type 1 diabetics.

As a result, there is a need for the development of superior therapies for the prevention and treatment of diabetes and obesity. To facilitate this need, there is a requirement for greater understanding of glucose uptake in skeletal muscles, specifically in muscles of diabetic and obese subjects. Further, there is a need for effective recommendations for diabetics and the obese with respect to nutrition and caffeine consumption. There is a further need to develop drugs that aid glucose uptake in cells, specifically for the prevention and treatment of diabetes and obesity.

### SUMMARY OF THE INVENTION

The present invention relates to the discovery that adenosine receptor antagonists inhibit the uptake of glucose in cells.

According to one embodiment of the present invention, there is provided a a diet plan method for a disease selected from the group consisting of diabetes and obesity wherein the diet plan method has a restricted amount of caffeine.

According to another embodiment of the present invention, there is provided a preventative diet plan method for a disease selected from the group consisting of Type 2 diabetes and obesity wherein the diet plan method has a restricted amount of caffeine.

According to another embodiment of the present invention, there is provided a diet plan method for a disease selected from the group consisting of diabetes and obesity wherein the diet plan method promotes the use of decaffeinated coffee.

According to another embodiment of the present invention, there is provided a preventative diet plan method for a disease selected from the group consisting of Type 2 diabetes and obesity wherein the diet plan method promotes the use of decaffeinated coffee.

According to another embodiment of the present invention, there is provided a caffeine-free appetite suppressants for diabetics which are caffeine free, low in sugars and labelled as “Diabetic Safe”.

According to another embodiment of the present invention, there is provided a caffeine-free weight loss products for diabetics which are caffeine free, low in sugars and labelled as “Diabetic Safe”.

According to another embodiment of the present invention, there is provided a caffeine-free energy drinks for diabetics which are caffeine free, low in sugars and labelled as “Diabetic Safe”.

According to another embodiment of the present invention, there is provided pharmaceutical compositions for diabetics which are caffeine free, low in sugars, and labelled as “Diabetic Safe”.

According to another embodiment of the present invention, there is provided pharmaceutical compositions wherein the compositions are remedies for the common cold.

According to another embodiment of the present invention, there is provided a system for health management of a disease selected from the group consisting of diabetes and obesity comprising counseling a patient regarding the consumption of non-caffeinated products and providing non-caffeinated products to the patient.

According to another embodiment of the present invention, there is provided a method for the prevention of a disease selected from the group consisting of Type 2 diabetes and obesity, in patients with a susceptibility to one of diabetes and obesity, comprising the steps of:

a. identifying patients with a susceptibility to one of diabetes and obesity,

b. counseling the patients regarding a restricted caffeinated diet, and

c. making available non-caffeinated products to the patients.

According to another embodiment of the present invention, there is provided a method of treatment for patients with a disease selected from the group consisting of diabetes and obesity, comprising

a counseling the patients regarding a restricted caffeinated diet, and

b making available non-caffeinated products to the patients.

According to another embodiment of the present invention, there is provided a method of treatment where the non-caffeinated products selected from the group consisting of caffeine-free coffee, colas, chocolate, energy drinks, pharmaceuticals and diet products.

According to another embodiment of the present invention, there is provided a use of decaffeinated coffee for the prevention of a disease selected from the group consisting of Type 2 diabetes and obesity.

According to another embodiment of the present invention, there is provided a use of decaffeinated coffee for the treatment of a disease selected from the group consisting of diabetes and obesity.

According to another embodiment of the present invention, there is provided a use of compounds selected from the group consisting of adenosine and adenosine receptor agonists. According to another embodiment of the present invention, there is provided a adenosine analogues, adenosine derivatives, adenosine conjugates and mixtures thereof as a treatment for a disease selected from the group consisting of diabetes and obesity.

According to another embodiment of the present invention, there is provided a use of compounds selected from the group consisting of adenosine, adenosine receptor agonists, adenosine analogues, adenosine derivatives, adenosine conjugates and mixtures thereof as a treatment for the prevention of a disease selected from the group consisting of Type 2 diabetes and obesity.

According to another embodiment of the present invention, there is provided a use of compounds selected from the group consisting of adenosine, adenosine receptor agonists, adenosine analogues, adenosine derivatives, adenosine conjugates and mixtures thereof as a method to offset the antagonistic effect of caffeine.

According to another embodiment of the present invention, there is provided a labeling system for diabetics and people susceptible to diabetes comprising labeling food and pharmaceutical products that are caffeine-free and low in simple sugars as safe for diabetics.

According to another aspect of the present invention, there is provided a sports drink comprising an adenosine receptor agonist.

### DESCRIPTION OF THE DRAWINGS

FIG. 1 is a graph showing the blood glucose response before and during oral glucose tolerance tests.

FIG. 2 is a graph showing the serum insulin response before and during oral glucose tolerance tests.

FIG. 3 is a graph showing the serum C peptide response before and during oral glucose tolerance tests.

FIG. 4 is a graph showing the effect of caffeine, coffee, decaffeinated coffee and placebo on glucose level during oral glucose tolerance tests.

FIG. 5 is a graph showing the effect of caffeine, coffee, decaffeinated coffee and placebo on insulin level during oral glucose tolerance tests.

FIG. 6 is a graph showing the effect of caffeine, coffee, decaffeinated coffee and placebo on C peptide level during oral glucose tolerance tests.

### DEFINITIONS

Male Sprague-Dawley rats: a breed of rats commonly used in research.

soleus muscle strips: soleus muscle from rats that is gently torn into two or three strips for the purpose of studying metabolism.

3MGT: 3-methyl glucose is a labelled glucose and is used to study glucose uptake or transport.

BMI: Body mass index

CPA: N6-cyclopentyladenosine (an adenosine A1 agonist).

DPMA: N-[2-(3,5-dimethoxy-phenyl)-2-(2-methylphenyl)ethyl]-adenosine is a drug that is an adenosine receptor agonist.

GLUT-4: the main glucose transporter in the cell.

P13 kinase: phosphatidylinositol 3-kinase.

IRTK: insulin receptor tyrosine kinase is an insulin signalling factor that promotes movement and translocation of GLUT 4 into cell membranes

IRS-1: insulin receptor substrate −1 is an insulin signalling factor that promotes movement and translocation of GLUT 4 into cell membranes.

MAP kinase: mitogen-activated protein kinase is an insulin signalling factor that promotes movement and translocation of GLUT 4 into cell membranes.

Akt: serine/threonine kinase.

### DETAILED DESCRIPTION OF THE INVENTION

One aspect of the present invention is that caffeine is detrimental to glucose uptake in human skeletal muscle cells of healthy subjects (i.e. non-diabetic lean males). Examples 1, 2, and 17 illustrate this aspect of the invention in more detail. This aspect of the invention demonstrates that caffeine ingestion affects glucose metabolism. Further, a caffeine-free diet is beneficial to insulin's action. Further still, compounds that act in the opposite manner to caffeine (i.e. adenosine receptor agonists) promote insulin's actions.

Another aspect of the present invention is that caffeine is detrimental to glucose uptake in human skeletal muscle cells of obese and diabetic subjects. Examples 5, 6, 7, 8, 9, 11, 16, 18 and 19 describe this aspect of the invention in more detail. The result is that diabetic and obese patients will make effective nutritional choices for the treatment of diabetes and obesity. Further, people with a susceptibility to diabetes and obesity can make effective nutritional choices for the prevention of Type 2 diabetes and obesity. For example, it is beneficial for diabetics to avoid caffeine. Caffeine is present in many “everyday” products such as coffee, tea, weight loss products, energy drinks, soft drinks and pharmaceuticals, especially remedies for the common cold. Diabetics should avoid caffeine-containing products, and if possible choose caffeine-free/caffeine-reduced weight loss products, energy drinks, soft drinks and pharmaceuticals. Further, diabetics, obese patients and those susceptible to diabetes and obesity should adopt a system of health management to avoid/minimize caffeine consumption. Further, another aspect of the present invention relates to a labeling system for diabetics and people susceptible to diabetes comprising labeling food and pharmaceutical products that are caffeine-free/caffeine-reduced and low in simple sugars as safe for diabetics.

Another aspect of the present invention is that decaffeinated coffee increases glucose uptake. Example 3 describes this aspect of the invention in more detail. Coffee is comprised of many compounds, caffeine constituting a mere 1-3% of the content. The benefits of this aspect of the invention are that (1) decaffeinated coffee contains a compound or compounds that aids glucose uptake, (2) consumption of decaffeinated coffee helps to treat or prevent diabetes and obesity, and (3) coffee is a biological source of an adenosine receptor agonist useful for increased uptake of glucose.

Another aspect of the present invention is that adenosine, adenosine analogues, derivatives and conjugates thereof promote glucose uptake in skeletal muscle in healthy, diabetic and obese subjects. Examples 3 and 16 describe this aspect of the invention in more detail. As a result, adenosine, adenosine analogues, derivatives and conjugates thereof can be used to prevent and treat diabetes and obesity.

Another aspect of the invention relates to the role of adenosine receptor antagonists during exercise. Examples 10 and 11 describe this aspect of the invention. As a result, the use of adenosine receptor agonists in sports drinks, is beneficial for getting glucose into the muscle in recovery from exercise.

Another aspect of the invention relates to the role of adenosine receptor antagonists on blood response to high and low index glycemic foods in non-diabetic and diabetic volunteers. Examples 17 and 18 describe this aspect of the invention. The result is that diabetic patients will make effective nutritional choices for the treatment of diabetes. Further, people with a susceptibility to diabetes can make effective nutritional choices for the prevention of Type 2 diabetes.

### Methods and Materials

The methods and materials employed in this invention are common to those skilled in the art. The methods and materials for the hyperinsulinemic euglycemic glucose clamp test are found in Greer et al., 2001. The methods and materials for the oral glucose tolerance test (OGTT) are described in Graham et al., 2001.

### EXAMPLES

### Example 1

### Caffeine Ingestion Decreases Glucose Disposal During Hyperinsulinemic Euglycemic Clamp in Humans

Nine, lean sedentary males underwent two hyperinsulinemic euglycemic clamp sessions, one following caffeine ingestion (5 mg/kg) and one following placebo (dextrose). Trials were separated by one week. Prior to each clamp session, subjects withdrew from methylxanthine containing products for 48 hours. Following caffeine ingestion, glucose disposal was 6.38+/−0.76 mg/kg/min compared with 8.42+/−0.63 mg/kg/min in the placebo. This represents a 24% decrease in glucose disposal following adenosine receptor antagonism by caffeine. Furthermore caffeine ingestion resulted in a 35% decrease in carbohydrate storage compared to placebo and is consistent with the decreased glucose uptake observed with caffeine administration. Since skeletal muscle is the most likely site for insulin-mediated glucose disposal, these data suggest that adenosine plays a role in regulating glucose uptake in human skeletal muscle.

### Example 2

### Caffeine Ingestion Increases Circulating In Humans During an Oral Glucose Tolerance Test

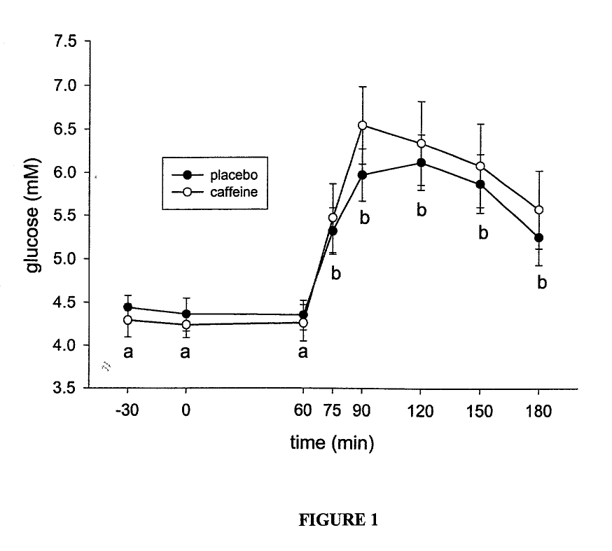

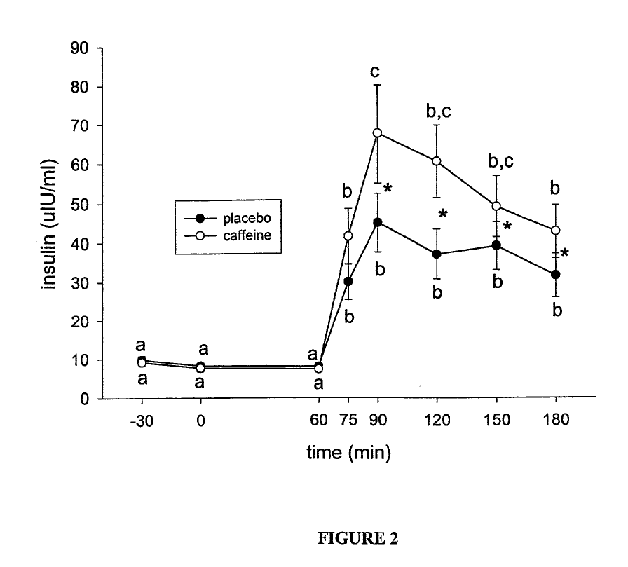

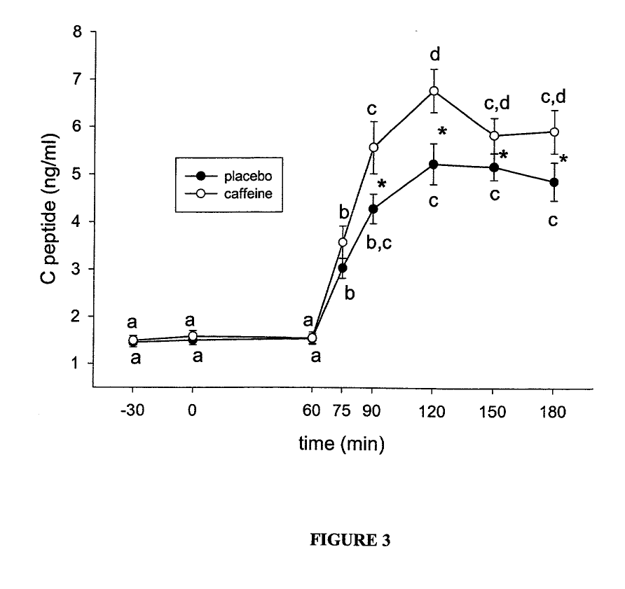

Young, fit, adult males (n=18) underwent two oral glucose tolerance tests (OGTT). The subjects ingested caffeine (5 mg/kg) or placebo (double blind) and one hour later, ingested 75 g of dextrose. Prior to the OGTT there were no differences between or within trials in circulating serum insulin, or C peptide, or blood glucose or lactate. Following the OGTT all of these parameters increased (p≦0.05) for the duration of the OGTT. Caffeine ingestion resulted in an increase (p≦0.05) in serum fatty acids, glycerol and plasma epinephrine. During the OGTT these decreased to match those of the placebo trial. In the caffeine trial the serum insulin and C peptide concentrations were significantly greater (p≦0.05) than for placebo for the last 90 minutes of the OGTT (see FIGS. 2 and 3) and the area under the curve for both measures were 60 and 37% greater (p≦0.05) respectively. This prolonged, greater elevation in insulin did not result in a lower blood glucose level (see FIG. 1); there were no differences between trials. The data support the hypothesis that caffeine ingestion impairs glucose disposal. Further, the data suggests this is due to adenosine receptor antagonism in skeletal muscle.

### Example 3

### Impaired Response to an Oral Glucose Tolerance Test Following Ingestion of Caffeine in Alkaloid Form or as a Component of Coffee

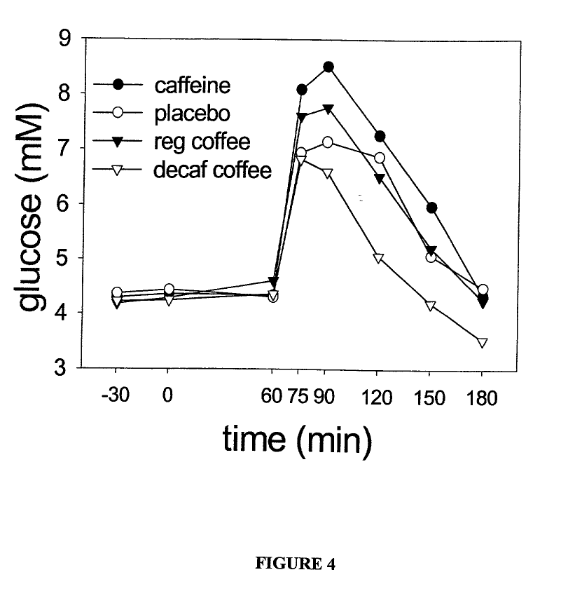

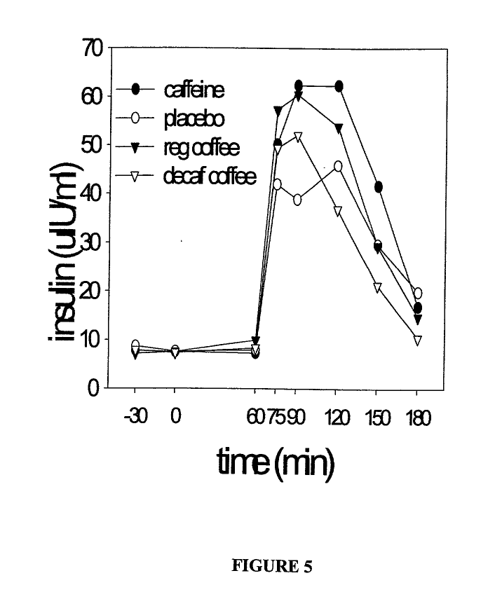

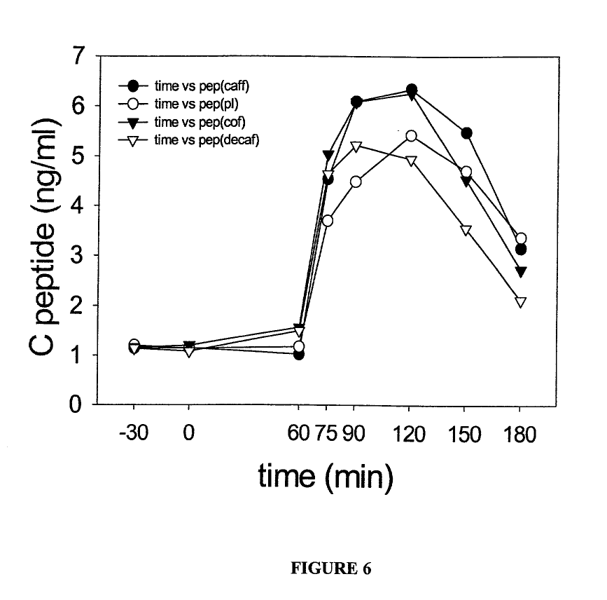

Ten healthy male subjects, who were not regular caffeine users, underwent an oral glucose tolerance test on four occasions following the ingestion of either pure alkaloid caffeine capsules (5 mg/kg) (AC), caffeinated coffee (CC), decaffeinated coffee, or placebo capsules (PL). Venous blood samples were taken at −30, 0 (treatment given), 60 (OGTT administered), 75, 90, 120, 150 and 180 minutes and were analyzed for glucose, insulin, C peptide, glycerol, free fatty acids and lactate. Area under the curve (AUC) were calculated for the two hours of the OGTT. As previously seen, AC demonstrated a higher AUC for insulin (see FIG. 5) than both PL (p<0.002) and DC (p<0.001) respectively. As well, insulin AUC for CC showed a similar trend to AC, approaching a significantly higher insulin AUC than DC (p<0.08). AUC for peptide C (See FIG. 6) demonstrated similar results to insulin, again with AC showing higher values than both PL (p<0.02) and DC (p<0.001) and CC approaching significantly higher values that DC (p<0.08). AUC for glucose with AC was higher than both CC (p<0.04) and DC (p<0.01) respectively and CC was higher that DC (p=0.05). As well, PL demonstrated higher AUC for glucose than DC (p<0.02) See FIG. 4. In conclusion, it appears than CC can elicit a similar insulin insensitivity as observed with AC, but to a lesser extent. These results also suggest that some component of coffee may enhance insulin-mediated glucose uptake in resting humans. This component acts as an adenosine receptor agonist.

### Example 4

### The Determination of Adenosine Receptors in Skeletal Muscle

The purpose of this study was to investigate the presence of A₁ and A₂ adenosine receptors (AR) in rat and human skeletal muscle. Adenosine receptor-stimulated adenylate cyclase activity studies were conducted to determine cAMP responses in the presence of AR agonists using rat skeletal muscle homogenates. Subsequently, Western blotting experiments were conducted to identify the A₁ and A₂ receptor proteins in rodent and human skeletal muscle samples. In the initial experiments, NECA (an A₂ AR agonist) produced a significant 7.5-fold increase in cAMP production in rat oxidative muscle homogenates with no effect on glycolytic samples. There was little effect of R-PIA (an A₁ AR agonist) on cAMP levels in any fiber type. Western blotting revealed A₁ and A₂ AR protein bands in rat and human skeletal muscle samples. A₁ and A₂ₐ expression was observed in similar amounts in both oxidative and glycolytic rodent fibers. A₂ₐ expression was highest in the liver compared to muscle and heart homogenates. Both rodent oxidative and glycolytic skeletal muscle homogenates had a significantly greater A₁ and A₂ expression compared to heart. In conclusion, both receptor subtypes appear to be present in oxidative and glycolytic muscle fibers in rodents and both receptor subtypes appear to be present in human skeletal muscle.

### Example 5

### Caffeine Ingestion Increases Circulating Insulin in Obese Males During an Oral Glucose Tolerance Test

Four young, obese (BMI>30) nondiabetic males, underwent an OGTT similar to Example 2. When lean males (n=18) underwent OGTT with and without caffeine ingestion the AUCs for GLU were 168 and 208 mM/2 h, respectively. The comparable data for the obese males were very similar (167 and 229 mM/2 h). However, there were marked differences for insulin: the lean males had AUCs of 3274 and 5242 uU/ml/2h for placebo and caffeine respectively, while comparable data for the obese males were 6938 and 10,968 uU/ml/2 h. The obese subjects had an insulin response to an OGTT that was over 100% greater than that of the lean subjects, and the insulin resistance induced by caffeine ingestion was twice as large.

### Example 6

### Effects of Caffeine Ingestion on the Insulin Response in Humans During an Oral Glucose Tolerance Test Before and After a Weight Loss Program

Six, obese (BMI=30-38 kg/m2) men performed an OGTT one hour after ingesting caffeine (5 mg/kg) or placebo (P). The two OGTT's were repeated after a twelve week nutrition-exercise intervention during which time subjects abstained from caffeine and lost 3-12 kg. There were no differences among the four trials in insulin, C-peptide or glucose prior to the OGTT. Prior to the twelve week intervention, caffeine ingestion resulted in a greater (p=0.043) insulin response during the OGTT, although there were no differences in blood glucose. Following the intervention there was no detectable change in the OGTT response for either placebo or caffeine. Similarly, caffeine still resulted in a greater (p=0.056) increase in insulin during the OGTT compared to placebo. The intervention successfully lowered body weight, but failed to improve the insulin response to glucose ingestion, and the caffeine ingestion continued to exaggerate this response.

### Example 7

### Effect of Caffeine on the Insulin/Glucose Response to an OGTT in Obese, Resting Males

Young, sedentary, obese males (n=28) underwent two OGTT's ingesting caffeine (5 mg/kg) or placebo followed by 75 g of dextrose one hour later. Prior to the OGTT there were no differences between or within trials in insulin or glucose levels. Caffeine resulted in significantly greater (p<0.05) glucose and insulin for the last 90 minutes and 105 minutes respectively of the OGTT. The area under the curve during the OGTT was greater following caffeine for both insulin (9455.8 -uIU/ml/2h (□˜640.7) and glucose (260.1 mM/2h (−22.4) in comparison to placebo (7037.9 uIU/ml/2h (□˜631.5 and 188.5 mM/2h (□˜25.3) respectively (p<0.05). The results indicate that caffeine ingestion may exaggerate insulin resistance associated with obesity.

### Example 8

### Characterization of the Impact of Adenosine Receptor Antagonism on Obese and Type 2 Diabetics, with Emphasis on the Ability of Caffeine to Generate Insulin Resistance.

Groups of obese (BMI>30) (n=18), and Type 2 diabetic (n=18) males, age 18-30 years old are undergoing two OGTTs, with and without caffeine ingestion one hour prior to the OGTT. The results will be compared to the current data base; containing data (28,40) collected in an identical fashion for 30 lean (BMI of less than 25) males age 18-30 years. Lean subjects have BMI of less than 25, the obese are class I (BMI 30-34), abdominal obese (waist circumference>100 cm) and the diabetics have the same anthropometry. The diabetics are volunteers who have been well controlled for a year, with glycosylated hemoglobin levels between 6.5-9.5% for at least three months. They have similar levels of obesity, are not insulin-dependent, and are not on oral hypoglycemic agents, but rather diet-controlled. Their participation is medically approved and they are screened for hypertension and angina. The area under the curve for insulin and C-peptide is greater for the obese and even greater for the diabetic subjects. Following caffeine ingestion the area under the curves for insulin and C-peptide are increased in the lean subjects. Caffeine causes an even greater response in these parameters for the obese and diabetic subjects. Data also includes plasma catecholamines, methylxanthines, FFA, and glycerol.

### Example 9

### Effect of Habitual Caffeine Ingestion on Glucose Uptake

The subjects of this trial are lean, obese and obese males with Type 2 diabetes (n=8 each, age 18-30 years) which have had their exercise habits, diet and caffeine consumption regulated. All subjects are sedentary (weekly activity of less than one hour). For one month prior to the study, subjects are monitored to establish that their exercise habits are regular, they are weight stable and their diet is energy, macronutrient and caffeine stable. They are monitored to ensure that they maintain these patterns for the following three months. In a double-blind, crossover design they consume either caffeinated or decaffeinated coffee for one month with the two trials separated by a one month ‘washout’ period. Subjects are provided with packets of ground coffee and brewing instructions. For the caffeinated treatment, the coffee packets deliver 4.5 mg/kg of caffeine in their coffee (2 mugs of coffee) twice a day. At 0, 1 and 4 weeks of each trial the subjects undergo an OGTT 1 hour following ingestion of 2 mugs of their prescribed coffee. No exercise is performed within two days of the OGTT and carbohydrate ingestion is regulated. Muscle biopsies are taken before and after each OGTT. Blood samples are analyzed and biopsies are analyzed for A1 and A2a receptor protein and mRNA, cAMP, glycogen and IRTK activity, IRS-1 associated PI3K, Akt and GSK-3. Prior to treatments, the obese and diabetic subjects are insulin resistive and have a greater insulin response to caffeine. Habitual ingestion of coffee upregulates A1 receptors progressively over the month. During the transition (i.e., at one week), a decrease in the insulin response is observed. Upon habituation (one month), the response returns to normal.

### Example 10

### Caffeine Impairs Glucose Uptake but not Insulin Signaling in Rested and Exercised Human Skeletal Muscle

The role of adenosine in regulating insulin-stimulated glucose uptake in human skeletal muscle is not known. We investigate the effects of caffeine, a non-selective adenosine receptor antagonist, on skeletal muscle glucose uptake during a 100 minute euglycemic-hyperinsulinemic (100 μU/ml) clamp. On two occasions, seven males performed one hour one-legged knee extensor exercise three hours before the clamp. Caffeine (5 mg/kg) or placebo was administered in a randomized, double blind fashion one hour before the clamp. Whole body glucose disposal was reduced (p<0.05) in caffeine (37.5+/−3.1 μmol/min/kg) vs. placebo (54.1+/−2.9 μmol/min/kg). Total (area under the curve) insulin-stimulated glucose uptake (arterio-venous concentration difference×blood flow) was higher (p<0.05) in the exercised (63.3+/−13.1 mmol/100 min) than rested (37.0+/−6.7 mmol/100 min) leg. However caffeine reduced (p<0.05) total insulin-stimulated glucose uptake equally in exercised (32.9+/−3.7 mmol/100 min) and rested (17.9+/−6.2 mmol/100 min) legs. Insulin increased insulin receptor tyrosine kinase (IRTK), insulin receptor substrate 1-associated phospatidylinositol 3-kinase (P13K) activities and serine phosphorylation of Akt significantly, but similarly in rested and exercised legs. Furthermore, insulin decreased glycogen synthase kinase-3 (GSK-3) activity equally in rested and exercised legs. However, caffeine had no effect on insulin-stimulated IRTK, P13K, AKT, or GSK-3 in relaxed or exercised legs. We conclude in humans 1) caffeine impairs insulin stimulated glucose uptake in rested and exercised skeletal muscles, and 2) caffeine-induced impairment of insulin-stimulated muscle glucose uptake is not accompanied by alterations in IRTK, P13K, Akt or GSK-3.

### Example 11

### Effect of Exercise Training on the Response to Adenosine Receptor Antagonism and its Association with Alterations in Glucose Management

Lean, sedentary males, and obese males with and without Type 2 diabetes (n=8 each) are performing a 12-week, exercise training program. Subjects are selected to have similar initial fitness levels (VO₂ max) and diet and caffeine habits are controlled throughout the study. During the pretreatment period, subjects consume a weight maintenance diet (55% carbohydrate, 20% protein, 25% fat). Their body composition is assessed for total adiposity as well as visceral and subcutaneous fat before and after the treatment by Magnetic Resonance Imaging (MRI). The exercise program is supervised and consists of walking or running on a treadmill at 40-60% of HR reserve for 60 minutes five times per week. Subjects increase their energy intake to keep their weight stable to minimize changes in adipose tissue. Thus, major alterations in metabolic responses are more likely attributed to training adaptations in muscle. Euglycemic-hyperinsulinemic clamp tests with and without caffeine are performed before and after the training program (those after the training are performed at least 48 hours after the last exercise). Muscle biopsies are taken before and after each clamp and analyzed. Insulin sensitivity increases as does A1 receptors. The relative improvement is greatest in the Type 2 diabetics and least in the lean subjects.

### Example 12

### Effect of Adenosine on Skeletal Muscle Glucose Transport

Male Sprague-Dawley rats are used in all the following examples. Soleus muscle strips are incubated as described by Bonen et al (1992), Wilkes and Bonen (2000) and Bonen et al (1994) to determine glucose transport. Basal and insulin-stimulated 3-O-methyl glucose transport (3MGT) is examined in a dose dependent (0-10 nM) manner during a 10 minute incubation period in appropriate buffer (Bonen et al., 1994). Comparison of this dose-response relationship of insulin, and the A1 agonist CPA, and A2 agonist DPMA on 3MGT is conducted. 3MGT rates are linear for up to 20 minutes. The 10 minute period is therefore a convenient time point to acquire sufficient counts in the muscle at a reasonable cost of using radiolabelled 3MGT.

Optimal stimulating concentrations of insulin, CPA, and DPMA are established for 3MGT. Insulin stimulated 3MGT is inhibited by LY29004, an inhibitor of PI3-kinase, 2) CPA- and DPMA-induced increases in 3MGT are also inhibited by LY29004, and 3) exposure to these stimulators alone or in combination have additive effect on 3MGT.

### Example 13

### Effect of Adenosine on Membrane-bound GLUT 4 Translocation and Intrinsic Activity

The optimal glucose transport stimulating concentrations of insulin, CPA and DPMA, are used to determine the increase, or lack thereof, in surface GLUT 4 using the method of radiolabelling the surface GLUT 4 with bis-mannose photolabel which has been used successfully by Bonen et al (1992), Han et al. (1998) and Wilkes and Bonen (2000) and others (Etgen et al., 1996; Lund et al., 1995). With this procedure ³H-bis-mannose (2-N-4(1-azi-2,2,2-trifluoroethyl)-benzoyl-1,3-bis-(D-mannose-4-yloxy)-2-propylamine) (ATB-[2-³H]BMPA) is provided to isolated muscles exposed to one of the treatments. The muscle is frozen (−80° C.), solubilized crude membranes are prepared (Han and Bonen, 1998) and GLUT 4 is then immunoprecipitated with affinity purified anti-GLUT 4 to separate it from surface GLUT 1 that is also labeled. SDS/PAGE is used to separate GLUT 4 and remaining proteins. Then the gel is cut into 4 mm slices which are solubilized and counted for ³H.

These experiments are designed to determine whether insulin and the A1 and A2 agonists translocate and/or activate GLUT 4. In many experiments, the fold increases in surface GLUT 4 and 3MGT by insulin and by contracting muscle are nearly identical (Etgen et al., 1996, Lund et al., 1995, Reynolds et al., 1997). Thus, by combining the observations on glucose transport and surface GLUT 4 one can ascertain whether 3MGT increments in muscle are due to increases in surface GLUT 4 (i.e. same fold increase in 3MGT and surface detectable GLUT 4). A mismatch in the fold increases in these responses, indicates that the activity of surface detectable GLUT 4 has been altered. This basic approach is commonly used to ascertain if there is a change in intrinsic activity of surface GLUT 4 (Bonen et al., 1992; Han and Bonen, 1998; Wilkes and Bonen, 2000; Hansen et al., 1998).

The demonstration of a 1:1 relationship between glucose transport and surface GLUT 4 accumulation by insulin, CPA and DPMA, leads to additional experiments to identify insulin-sensitive and CPA and DPMA-sensitive intracellular GLUT 4 pools. Han and Bonen (1998) and Lemieux et al.(2000) have experience with various muscle fractionation procedures and identification of intracellular GLUT 4 pools using a variety of marker proteins. These experiments potentially reveal novel means of stimulating GLUT 4 translocation from insulin-insensitive pools.

### Example 14

### Determination of the Signaling Proteins Associated with GLUT 4 Translocation which are Activated by Adenosine

Experiments with insulin, CPA and DPMA are performed in isolated muscles using optimal stimulating concentrations to determine which signaling proteins are activated. For this purpose, isolated rodent muscles are incubated with either basal or maximal insulin concentrations with and without either CPA or DPMA. Glucose transport is determined by 3MGT, measured over 10 minutes. The muscle samples are analyzed for IRTK, IRS-1 associated P13 kinase, p38 MAP kinase and Akt activities. The critical exposure time to activate signaling proteins is established in pilot work. Wilkes et al. (2000a) and Wilkes et al. (2000 b) have found that 5 minute exposure provides the optimal period for detecting signaling protein activation for insulin in incubated muscles. Lemieux et al (2000) have recently found that there are several intracellular GLUT 4 pools that are independently activated, thus it is conceivable that CPA and DPMA may recruit GLUT 4 from one of these ‘non-insulin sensitive’ pools. Optimal exposure times required for signaling protein activation by CPA and DPMA are established. We do not assume that these are the same as for insulin. We establish that blocking P13-kinase with LY29004 blocks downstream activation of Akt by insulin, and by CPA and DPMA.

### Example 15

### The Impact of Overexpressing A1 or A2a Receptors on Muscle Glucose Uptake

Electroporation, a method of non-viral gene transfer uses electric pulses (electroporation) to transfect prokaryotic and eukaryotic cells in vitro. Mir et al. (1999) demonstrated conclusively that it is possible to transfer plasmid DNA into skeletal muscle in vivo by using electroporation. The extent of the transfection is dependent on the voltage selected, pulse duration, number of pulses and their frequency. Thus, it appears that electroporation increases gene transfer into muscle not only by muscle-fiber permeabilization but also by the direct effect on the DNA molecule (Mir et al., 1999). They have demonstrated i) the very large increase in gene expression that could be attained, ii) the long-term stability of the effect (i.e. up to nine months), iii) the reduction in variability, iv) its application to a variety of species (mouse, rat, rabbit, monkey), and v) being able to regulate the degree of expression (Mir et al., 1999).

We are using the electroporation and cDNA injection procedures of Mir et al. (1999) to overexpress either the A1 or the A2a receptor alone or in combination in soleus muscle. The contralateral leg (sham electroporation) serves as control. We examine insulin, CPA and DPMA-stimulated 3MGT, insulin signaling and the GLUT 4 responses in the isolated soleus muscles that contain the upregulated proteins. These experiments establish whether the increased availability of either A1 or A2a receptors, alone or in combination, further increase insulin, CPA and DPMA-stimulated 3MGT, and GLUT-4 translocation or activation, by means of procedures described in experiments outlined above.

### Example 16

### Characterization of the Impact of an Adenosine Receptor Agonist in Obese and Type 2 Diabetics, with Emphasis on the Ability of Adenosine Receptor Agonist to Increase Glucose Uptake

Groups of obese (BMI>30) (n=18), and Type 2 diabetic (n=18) males, age 18-30 years undergo two OGTT's, with and without adenosine receptor agonist ingestion one hour prior to OGTT. Venous blood samples are taken at −30, 0 (treatment given), 60 (OGTT administered), 75, 90, 120, 150 and 180 minutes and are analyzed for glucose, insulin, C peptide, glycerol, free fatty acids and lactate. Area under the curve are calculated for the 2 hours of the OGTT. The obese subjects are class 1 (BMI 30-34), abdominal obese (waist circumference>100 cm) and the diabetics have the same anthropometry. The diabetics are volunteers who have been well controlled for a year, with glycosylated hemoglobin levels between 5.4-9.9% for at least three months. They have similar levels of obesity, are not insulin-dependent, and are not on oral hypoglycaemic agents, but rather diet-controlled. Their participation is medically approved and they are screened for hypertension and angina.

### Example 17

### Effect of Caffeinated Coffee on the Insulin and Glucose Responses to Either a High or Low Glycemic Index Breakfast Cereal in Lean, Resting Males

To date the majority of studies investigating caffeine and insulin resistance have compared pure caffeine with a standard 75 g oral dextrose load (OGTT) against a placebo treatment. The current study investigated whether impaired glucose management occurs with normal foods, namely following ingestion of coffee and either a high or low glycemic index (GI) breakfast cereal. Young (age=24±2 year), non-obese (BMI=25±1 kg/m²), non-diabetic males (n=6) underwent four separate trials approximately one week apart, in a randomized order. The four treatments were: (1) caffeinated (5 mg/kg) coffee with either a high glycemic index or (2) low glycemic index cereal or (3) decaffeinated coffee with either a high glycemic index or (4) low glycemic index cereal. Cereal with 150 ml skim milk resulted in 75 g total carbohydrate for both the high (GI=81) and low (GI=41) glycemic index meals. This amount of carbohydrate was chosen to provide the same amount of carbohydrate (75 g) as that used in our previous caffeine and OGTT studies in lean and obese males. Venous blood samples were taken prior to ingestion of coffee (t=−60 minutes) and cereal (t=0 minutes) and at t=15, 30, 45, 60, 90 and 120 minutes after cereal ingestion.

There were no differences among the four trials in insulin, C-peptide or glucose prior cereal ingestion. Insulin and C-peptide area under the curves were significantly increased with caffeinated coffee (p<0.05). Specifically, after the high glycemic index meal, insulin area under the curve increased 93% with caffeinated (3039±413 uIU/ml/2h) vs decaffeinated (1574±257 uIU/ml/2h) coffee, while after the low glycemic index meal, it was 69% higher with caffeinated (1449±194 uIU/ml/2h) vs decaffeinated (856±148 uIU/ml/2h) coffee. Similarly, C-peptide area under the curve increased 54% with caffeinated (567±79 ng/ml/2h) vs decaffeinated (369±48 ng/ml/2h) coffee after the high glycemic index meal and was 39% higher with caffeinated (258±42 ng/ml/2h) vs decaffeinated (185±12 ng/ml/2h) coffee after the low glycemic index meal. The increased insulin and C-peptide responses with caffeinated coffee occurred with significantly elevated blood glucose (p<0.05). Specifically, after the high glycemic index meal, glucose area under the curves were 186±46 and 14±20 mM/2h with caffeinated and decaffeinated coffee, respectively, while after the low glycemic index meal, glucose results were 88±23 and 5.3±14 mM/2h with caffeinated and decaffeinated coffee, respectively.

This study was the first to examine the effects of caffeine on blood glucose management using coffee and a more typical breakfast meal as opposed to pure caffeine and an OGTT. The results of the current study support our previous findings in that the ingestion of caffeinated coffee (equivalent to 5 mg caffeine per kg body weight) prior to a 75 g carbohydrate load (breakfast cereal) resulted in impaired glucose management in lean, healthy males. Overall, insulin, C-peptide, and glucose responses were elevated and prolonged with caffeinated coffee and either a high or low glycemic index cereal. Furthermore, the results suggest that the caffeine-induced impairment in blood glucose management was more pronounced after ingestion of the high glycemic index cereal, which represents a very common type of breakfast cereal consumed in the general population.

### Example 18

### Effect of Caffeine on Blood Glucose Response to a High Glycemic Index Cereal in Young Type 1 Diabetics

Young type 1 diabetics (5 females and 1 male) who had been diagnosed for an average of 8.3±5.4 years volunteered to perform two trials in a randomized, double blind study design. Subjects were given either placebo or caffeine-containing (5 mg/kg) capsules and 30 minutes later they self-administered the appropriate amount of insulin for the ingestion of 60 g of carbohydrates in the form of a high glycemic cereal with 125 ml of 1% milk. The amount of self-administered insulin and injection site were the same for both trials. Venous blood glucose was measured prior to capsule ingestion, prior to insulin injection, and immediately following ingestion of the cereal meal (t=0 minutes). Blood glucose was then measured every 15 minutes for the next 2.5 hours. The 72% difference in the glucose area under the curve for the caffeine and placebo trials (598±7.4 and 347±7.4 mM/2.5 h, respectively) were significantly different (p<0.01). The results from this study strongly suggest that caffeine ingestion caused a substantial impairment in glucose management which resulted in type 1 diabetics experiencing a high and prolonged elevation in blood glucose. These findings have important implications for type 1 diabetics who consume caffeine since individuals with type 1 diabetes lack the ability to produce endogenous insulin and thus must self-monitor their blood glucose levels and insulin requirements throughout the day.

### Example 19

### The Effect of Caffeine on Glucose and Insulin Responses in Obese Individuals with Type 2 Diabetes

Obese (BMI=32±1 kg/m²) type 2 diabetic males (n=8, age=46±2 year) underwent two OGTTs, with and without caffeine (5 mg/kg) one hour prior to ingestion of 75 g of dextrose. Subjects had an average glycosylated hemoglobin level of 7.8±0.1%, had no diabetes-related visual or renal complications, and were not taking insulin to control their diabetes. Subjects were required to abstain from caffeine-containing food and beverages, alcohol, exercise and oral hypoglycemic medication for 48 hours prior to each trial. Venous blood samples were taken prior to ingestion of caffeine or placebo (time=−60 minutes) and the 75 g glucose load (t=0 minutes) with subsequent samples taken at t=15, 30, 60, 90, 120, 150 and 180 minutes after glucose ingestion. Based on our previous findings in obese males, the current protocol with obese type 2 diabetic subjects was designed to monitor blood glucose levels (as well as other blood metabolites) for an additional hour beyond the standard OGTT protocol we had previously used (i.e. 3 hour instead of 2 hour post glucose ingestion).

Average fasting blood glucose was 6.7±0.3 mM and there were no differences between or within trials in blood glucose levels prior to ingestion of the oral glucose load. Ingestion of caffeine resulted in significantly elevated (p<0.05) blood glucose for the last hour of the OGTT (from 120 to 180 minutes following glucose ingestion) when compared with placebo. Furthermore, when subjects ingested caffeine, blood glucose remained significantly elevated (p<0.05) at 180 minutes following ingestion of the OGTT (8.4±0.8 vs 7.4±1.0 mM for caffeine and placebo, respectively) compared with baseline values.

Thus, our study investigating the influence of caffeine followed by an OGTT in obese type 2 diabetics suggests that caffeine ingestion leads to elevated and prolonged blood glucose levels in these individuals. This caffeine-induced effect on blood glucose levels is comparable to our previous findings in both lean and obese non-diabetic subjects and provides further support for a negative impact of caffeine on blood glucose management. Furthermore, we have now demonstrated that caffeine ingestion creates a situation (i.e hyperglycemia) in which there are greater demands on insulin secretion in those already experiencing insulin resistance, such as obese type 2 diabetics. Overall, our results suggest that an individual with type 2 diabetes will spend a prolonged period of time in a hyperglycemic state following ingestion of caffeine and an oral glucose load. This negative impact of caffeine may have important implications since prolonged hyperglycemia is associated with a variety of long-term complications, such as microvascular, renal and visual complications.

### Pharmaceutical Compositions

Pharmaceutical compositions of the above compounds are used to treat patients that are obese and or have diabetes. Vehicles for delivering the compounds of the present invention to target tissues throughout the human body include saline and D5W (5% dextrose and water). Excipients used for the preparation or oral dosage forms of the compounds of the present invention include additives such as a buffer, solubilizer, suspending agent, emulsifying agent viscosity controlling agent, flavour, lactose filler, antioxidant, preservative or dye. There are preferred excipients for parenteral and other administration. These excipients include serum albumin, glutamic or aspartic acid, phospholipids and fatty acids.

The preferred formulation is in liquid form stored in a vial or an intravenous bag. The compounds of the present invention may also be formulated in solid or semisolid form, for example pills, tablets, creams, ointments, powders, emulsions, gelatin capsules, capsules, suppositories, gels or membranes.

Acceptable routes of administration include intravenous, oral, topical, rectal, parenteral (injectable), local, inhalant and epidural administration. The compositions of the invention may also be conjugated to transport molecules or included in transport modalities such as vesicles and micelles to facilitate transport of the molecules. Methods for the preparation of pharmaceutically acceptable compositions that can be administered to patients are known in the art.

The compositions of the invention may also be conjugated to transport molecules, monoclonal antibodies or transport modalities such as vesicles and micelles that preferentially target recipient cells.

Pharmaceutical compositions including the compounds of the present invention can be administered to humans and animals. Dosages to be administered depend on individual patient condition, indication of the drug, physical and chemical stability of the drug, toxicity, the desired effect and on the chosen route of administration. These pharmaceutical compositions are used to treat obesity and diabetes.

Although the invention has been described with preferred embodiments, it is to be understood that modifications may be resorted to as will be apparent to those skilled in the art. Such modifications and variations are to be considered within the purview and scope of the present invention.

All publications, patents, and patent applications are herein incorporated by reference in their entirety to the same extent as if each individual publication, patent or patent application was specifically and individually indicated to be incorporated by reference in its entirety.

### References

Battram, D. S., Rowland, M., Marko, N., Graham, T. 1999. Cdn. J. Appl. Physiol. 24: p. 425

Bonen, A, M G Clark, and E J Henriksen. Am J Physiol 266: E1-E16, 1994.

Bonen, A., L A Megeney, S C McCarthy, J C McDermott, and M H Tan. Biochem Biophys Res Comm 187: 685-691, 1992.

Brundin, T, R Branstrom, and J Wahren. Am J Physiol 271 :E496-E504, 1996.

Budohoski, L, R A J Challiss, B McManus, and E A Newsholme. FEBS 167:1-4, 1984.

Carey, G B. J Appl Physiol 88:881-887, 2000.

Carey, G B And K A Sidmore. Int J Obesity 18: 155-160, 1994.

Challiss, R A J, L Budohoski, B McManus, and E A Newsholme. Biochem J 221:915-917, 1984.

Challiss, R A J, S J Richards, and L Budohoski. Eur J Pharmacol 226:121-128, 1992.

Cortright, R N and G L Dohm. Can J Appl Physiol 22:519-530, 1997.

Crist, G H, B Xu, K F LaNoue, and C H Lang. FASEB J 12:1301-1308, 1998.

Dixon, A K, A K Gubitz, D J S Sirinathsinghji, P J Richardson and T C Freeman. Br J Pharmacol 118:1461-1468, 1996.

Etgen Jr, G J, C M Wilson, J Jensen, C W Cushman, and J L Ivy. Am J Physiol 271: E294-E301, 1996.

Fredholm, B B. Pharmacol Toxicol 76:93-101, 1995.

Graham, T E and K B Adamo. Can J Appl Physiol 24:393-415, 1999.

Graham, T. E., Sathasivam, P., Rowland, M., Marko, N., Greer, F., Battram, D. 2001 Can. J. Physiol. Pharmacol. (in press).

Green, A. J Biol Chem 262:15702-15707, 1987.

Greer, F., Hudson, R. Ross, R., Graham, T. 1998. Clinical and Investigative Medicine 83(Suppl)

Greer, F., Hudson, R., Ross, R., Graham, T. 2001. Diabetes (in press)

Han, X X, and A Bonen. Am J Physiol. 274: E700-E707, 1998.

Han, D-H, P A Hansen, L A Nolte and J O Holloszy. Diabetes 47:1671-1675, 1998.

Hansen, P A, W Wang, B A Marshall, J O Holloszy, and M Mueckler. J Biol Chem 273:18173-79, 1998.

Jequier, E and L Tappy. Physiol Rev 79:451-480, 1999.

Laybutt, D R, D J Chisholm and E W Kraegen. Am J Physiol 273:E1-E9, 1997.

Lemieux, K, X X Han, L Dombrowski, A Bonen, and A Marette. Diabetes 49: 183-189, 2000.

Lund, S, G D Holman, O Scmitz, and O Pedersen. PNAS 92: 5817-5821, 1995.

Meitzer, S, L Leiter, D Daneman, H Gerstein, D Lau, S Ludwig, J-F Yale, B Zinman, and D Lillie. Can Med Assoc J 159 (8 Suppl) 51-58, 1998.

Mauriege, P, D Prud'homme, S Lemieux, A Tremblay, and J P Despres. Am J Physiol 269:E341E-350, 1995.

Mir, L, M F Bureau, J Gehl, R Rangara, D Rouy, J M Caillaud, P DElaere, D Branellec, B Schwartz, and D Scherman. PNAS 96: 4262-4267, 1999.

Ralevic, V and G Burnstock. Pharmacol Rev 50:418-496,1998.

Reynolds IV, T H, J T Brozinick, M A Rogers, and S W Cushman. Am J Physiol 272: E320-E325, 1997.

Shaw, J E, P E Zimmet, D McCarty, and M de Courten. Diabetes Care 23:B5-B10, 2000.

Shryock, J C and L Belardinelli. Am J Cardiol 79:2-10, 1997.

Takasuga, S, T Katada, M Ui, and O Hazeki. J Biol Chem 274:19545-50, 1999.

Tan, M-H and D R MacLean. Clin Invest Med 18:240-246, 1995.

Taylor, R, T B Price, L D Katz, R G Shulman, and G I Shulman. Am J Physiol 265:E224-E229, 1993.

Vergauwen, L, P Hespel, and E A Richter. J Clin Invest 93:974-981, 1994.

Webster, J M, L Heseltine, and R Taylor. Biochim Biophys Acta 1316:109-113, 1996.

Wilkes, J J, and A Bonen. Am J Physiol. in press, 2000.

Wilkes, J J, R C Bell, and A Bonen FASEB J 14:A91, 2000.

Wilkes, J J, S Rodriguez, R C Bell, and A Bonen. FASEB J 14:A490, 2000.

Xu, B, D A Berkich, G H Crist, and K F LaNoue. Am J Physiol 274:E271-E279, 1998.

Zhang, Y and J N Wells. J Pharmacol Exp Therap 254:254:757-763, 1990.

We claim:

1. A diet plan method for a disease selected from the group consisting of diabetes and obesity wherein the diet plan method has a restricted amount of caffeine.

2. The diet plan method of claim 1 that is a preventative diet plan method.

3. A diet plan method for a disease selected from a group consisting of diabetes and obesity wherein the diet plan method promotes the use of decaffeinated coffee.

4. The diet plan method of claim 3 that is a preventative diet plan method.

5. Caffeine-free products for diabetics which are caffeine free, low in sugars and labelled as “Diabetic Safe”.

6. The caffeine-free product of claim 5 that is an appetite suppressant.

7. The caffeine-free product of claim 5 that is weight loss product.

8. The caffeine-free product of claim 5 that is an energy drink.

9. The caffeine-free product of claim 5 that is a pharmaceutical composition.

10. The pharmaceutical composition of claim 9 wherein the composition is a remedy for the common cold.

11. A system for health management of a disease selected from the group consisting of diabetes and obesity comprising counseling a patient regarding the consumption of non-caffeinated products and providing non-caffeinated products to the patient.

12. A method for the prevention or treatment of a disease selected from the group consisting of Type 2 diabetes and obesity, in patients with a susceptibility to one of diabetes and obesity, comprising the steps of:
a. identifying patients with a susceptibility to one of diabetes and obesity,
b. counseling the patients regarding a restricted caffeinated diet, and
c. making available non-caffeinated products to the patients.

13. The method of claim 12 where the non-caffeinated products selected from the group consisting of caffeine-free coffee, colas, chocolate, energy drinks, pharmaceuticals and diet products.

14. Method of using decaffeinated coffee for the prevention or treatment of a disease selected from the group consisting of Type 2 diabetes and obesity.

15. Method of using compounds selected from the group consisting of adenosine, adenosine receptor agonists, adenosine analogues, adenosine derivatives, adenosine conjugates and mixtures thereof as a treatment or prevention for a disease selected from the group consisting of diabetes and obesity.

16. Method of using compounds selected from the group consisting of adenosine, adenosine receptor agonists, adenosine analogues, adenosine derivatives, adenosine conjugates and mixtures thereof as a method to offset the antagonistic effect of caffeine.

17. A labeling system for diabetics and people susceptible to diabetes comprising labeling food and pharmaceutical products that are caffeine-free and low in simple sugars as safe for diabetics.

18. A sports drink comprising an adenosine receptor agonist.
